(19) United States
(12) Patent Application Publication
(10) Pub. No.: US 2017/0333359 A1
(43) Pub. Date: Nov. 23, 2017

(54) A DRUG-CONTAINING MICRO PARTICLE

(71) Applicant: RX ANALYTIC, INC, Ridgefield, CT (US)

(72) Inventor: Michael Goldstein, Ridgefield, CT (US)

(21) Appl. No.: 15/519,964

(22) PCT Filed: Oct. 20, 2015
(86) PCT No.: PCT/US15/56506

§ 371 Date: Apr. 18, 2017

Related U.S. Application Data
(60) Provisional application No. 62/066,326, filed on Oct. 20, 2014.

(51) Int. Cl. A61K 9/50

(52) U.S. Cl. CPC A61K 9/501; A61K 9/5089

(57) ABSTRACT

A micro particle, suitable for use in pharmaceuticals, is comprised of a drug particle having a size within the range of 1-10 microns and a metallic coating overlying the drug particle. The metallic coating can modulate the release of the drug from the micro particle. The metallic coating can be applied by physical vapor deposition (PVD).

This application claims the benefit of priority to prior U.S. Provisional application Ser. No. 62/066,326, filed Oct. 20, 2014; the entire contents of which are incorporated herein by reference.

### BACKGROUND OF THE INVENTION

The present invention relates to micro particles that contain a drug, to dosage forms containing them, and to methods of making the same.

Various pharmaceutical dosage forms are produced using drugs in the form of micro particles. Inhalable dosage forms, for example, frequently use drug micro particles carried by a gas; i.e., as an aerosol. Inhalable dosage forms can be used for treating the lungs per se and/or for systemic delivery. Typically the drug is in an immediate release form. However, some inhalable formulations have been proposed with a controlled or sustained release carrier.

For example, U.S. Pat. No. 6,254,854 relates to porous particles especially for deep lung delivery of a drug. The particles are typically made from a biodegradable polymer and have a mass density of less than 1.0 g/cm³, preferably less than about 0.4 g/cm³, and have a mean diameter of from about 100 nm to 15 um. The drug is trapped or encapsulated within the particle, such as during particle formation. The biodegradation of the polymer can provide a prolonged and/or controlled rate of drug delivery.

U.S. Pat. No. 6,942,868 relates to aerodynamically light particles for drug delivery to the pulmonary system. The particles are typically made of biodegradable polymer and have a tap density of less than 0.4 g/cm³ and a mean diameter between 5 nm and 30 nm. The drug is incorporated within the polymer particle. The release (or delivery) of the drug can be controlled by the type of biodegradable polymer.

U.S. Pat. No. 7,052,678 relates to inhalation particles having sustained release properties. The particles comprise a polycationic complexing agent that is complexed with a bioactive agent having a negative charge. Examples of the polycationic complexing agent include protamine, spermine, spermidine, chitosan, and polycationic polyamino acid. The bioactive agent includes therapeutic, prophylactic, and diagnostic agents. The complex of polycation and bioactive agent can be combined with pharmaceutically acceptable carriers, such as phospholipids, sugars and polysaccharides. The carrier material can provide advantageous particle characteristics for inhalation.

While several micro particle designs are known for delivering a drug, it would be advantageous to provide an alternative design.

### SUMMARY OF THE INVENTION

The present invention relates to a micro particle, comprising a drug particle having a size within the range of 1-10 microns and a metallic coating overlying the drug particle; wherein the drug particle comprises a biologically effective agent; and wherein the metallic coating comprises at least one metal. The metal can be contained in the metallic coating as elemental metal or as metal-containing compounds. The metallic coating is generally very thin, typically having an average thickness in the range of 1-25 nm. The biologically effective agent includes active pharmaceutical ingredients as well as nutrients and diagnostic agents. The metallic coating can provide an advantage to the drug micro particle, including the possibility of controlled, sustained, or delayed release of the drug.

Another aspect of the invention relates to a powder that comprises a plurality of the micro particles as described above and/or hereinafter. The powder is typically suitable for making a pharmaceutical dosage form such as an inhalable aerosol or a parenteral composition by the addition of a gas or sterile aqueous liquid. The powder may also contain excipients to facilitate the final dosage form or its administration.

A further aspect of the invention relates to a method, which comprises forming a metallic coating on a drug particle by a physical vapor deposition process, wherein the drug particle has a particle size of 1-10 microns and comprises a biologically effective agent and wherein the metallic coating comprises at least one metal. The physical vapor deposition process can be ion beam sputtering, magnetron sputtering, or evaporative sputtering. The metallic coating is typically applied to an average thickness of 1-25 nm.

### BRIEF DESCRIPTION OF THE DRAWINGS

FIG. 1 shows a first process flow chart of a method of making the micro particles of the present invention.

FIG. 2 shows a second process flow chart of a method of making the micro particles of the present invention.

FIG. 3 illustrates a piezoelectric atomizer for forming drug particle droplets with a charge that can be zero, negative, or positive.

FIG. 4 illustrates a physical vapor deposition chamber that can be used to form the metallic coating layer on the drug particles in-line with their production as illustrated in the first process flow chart or after said drug particles have been produced.

FIG. 5 illustrates a physical vapor deposition chamber that can be used to form the metallic coating layer on the drug particles after their production as illustrated in the second process flow chart.

### DETAILED DESCRIPTION OF THE INVENTION

The present invention relates to a micro particle comprising a drug particle of 1-10 microns having a metallic coating thereon. The metallic coating can modify the release of the drug from the micro particle and/or protect the drug. Accordingly, the use of these micro particles can provide a formulation that extends the release and/or effect of the drug, or, enhances the efficacy of the drug.

The drug particle used in the present invention contains a biologically effective agent that can provide therapeutic, prophylactic, or diagnostic action/activity. The drug particle may contain two or more biologically active agents. Also, the drug particle may contain non-active agents, such as pharmaceutical excipients. In most embodiments, however, the drug particle is primarily or exclusively a biologically effective agent or agents. That is, the drug particle may be comprised of at least 80%, typically at least 85%, more typically at least 90%, and often at least 95% by weight, of the biologically effective agent(s). It is possible, however, that the drug particle contain as little as 20% of the biologically effective agent(s). More typically the drug particle comprises 50-100% of the biologically effective agent(s), with amounts at or near 100%, e.g., 98%, 99%, being most common.

The biologically effective agents are not particularly limited and include pharmaceuticals, also known as active pharmaceutical ingredients or API's; vitamins; herbs; and markers such as chemical-, radio-, or imaging/contrast-markers. The pharmaceuticals can be bronchodilators, vasodilators, anti-inflammatories (steroidal and non-steroidal (NSAIDs), antibiotics, antivirals, mucolytics, cytotoxic agents, etc., but are not limited thereto. Pharmaceuticals that are useful in treating respiratory and/or lung disorders include: (1) short acting β adrenergic receptor agonists such as albuterol, levalbuterol, pirbuterol, fenoterol, epinephrine, ephedrine, terbutaline, and pharmaceutically acceptable salts thereof; (2) long acting β adrenergic receptor agonists such as salmeterol, clenbuterol, formoterol, bambuterol, indacaterol, and pharmaceutically acceptable salts thereof; (3) anticholinergic agents such as ipratropium, tiotropium, and their pharmaceutically acceptable salts (notably the bromine salt); (4) corticosteroids such as prednisone, prednisolone, beclomethasone, flunisolide, fluticasone, triamcinolone, budesonide, and pharmaceutically acceptable salts thereof; (5) antibiotics including tobramycin, colistimethate, gentamicin, amikacin, azetreonam, taurolidine, and pharmaceutically acceptable salts thereof; and (6) chemotherapeutics including those containing Platinum compounds (platins) used for cancer treatment, often lung cancer, such as Cisplatin, Carboplatin, Oxaliplatin, Satraplatin, Picoplatin, Nedaplatin, Triplatin, and Lipoplatin. A combination of biologically effective agents, especially combinations of two or more actives within one of groups (1)-(6) and/or a combination of actives from two or more of groups (1)-(6), can be present in a single drug particle. More typically, however, a combination of actives is achieved by combining different drug particles having different actives, which is explained more fully below regarding micro particle populations, powders, and pharmaceutical compositions.

The non-active agents, if present in the drug particle, are generally pharmaceutically acceptable excipients. Such excipients are typically a binder, carrier, and/or crystallization inhibitors. While a polymer can be incorporated into the drug particle, it is generally contemplated to limit or exclude the use of polymer matrices. For instance, the amount of biodegradable polymer is generally less than 10% the weight of the drug particle and preferably is not present in the drug particle. Likewise the amount of any polymer in the drug particle is typically less than 10% by weight, more typically less than 5% by weight, and usually 0% by weight of the drug particle.

As mentioned above, the drug particle has a particle size of 1 to 10 microns. In some embodiments it is 2-5 microns. The shape of the drug particle is generally spherical, but is not limited thereto. The micro particles of the invention comprise the drug particle overlain with a metallic coating.

The “metallic coating” as used herein means a coating that contains metal. The metal can be elemental metal, charged or ionic forms thereof, or a compound containing a metal, such as a metal oxide, a metal salt, or a metal-organic compound or complex. Generally the metal-containing compound must be conductive. For simplicity, reference hereinafter to a “metal” includes the element as well as the ions and compounds thereof unless otherwise indicated. The metallic coating of the present invention must contain at least one metal selected from the transition metals, alkaline earth metals, or alkali metals. For clarity, the “transition metals” refers to the metallic elements in groups 3-12 of the periodic table, also sometimes referred to as the d-block. Alkaline earth metals are in group 2 of the periodic table and include for example Magnesium and Calcium. Alkali metals are in group 1 of the periodic table and include for example Sodium and Potassium. Two or more metals can be used. The metal can be magnetic such as iron or its oxides, or non-magnetic (including paramagnetic and diamagnetic) such as gold or silver, etc. Usually non-magnetic metals are preferred. In many embodiments the metallic coating contains gold, silver, platinum, palladium, copper, nickel, or a combination of two or more thereof, with gold, silver, and platinum generally being the most preferred metals. Typically the metal is in elemental or ionic form. When the metallic coating contains other materials as part of a metal compound or as separate compounds, generally the total weight of the dominant metal in the metallic coating is at least 50% by weight, more typically at least 65% by weight, and still more typically at least 80% by weight, based on the total weight of the metallic layer. By mole, the total amount of the atoms of the metal in the metallic coating is generally at least 20%, typically at least 40%, more typically at least 60%, and often at least 80% or 90%, based on the total moles in the metallic coating. For clarity, a metallic coating made of elemental gold would mean theoretically that the total of the metal in the metallic coating was 100% by weight and 100% by mole. As a practical matter, however, impurities may slightly decrease the percentage, but generally at least 95%, at least 97%, at least 98%, and at least 99% are typically achieved, both by weight and mole, when coating an elemental metal or combination of elemental metals.

The metallic coating typically is comprised of, or consists only of, gold, silver, and/or platinum metals. The gold can be elemental gold, including charged forms thereof, a gold salt such as gold hydroxide or gold chloride, or a gold compound such as aurothioglucose. The silver can be elemental silver, including charged forms thereof, a silver salt, such as silver bromide or silver iodide, or a silver compound, such as silver nitrate or silver nitride. Generally any gold- or silver-containing salts/compounds can be used that is conductive. For simplicity, however, elemental gold, elemental silver, or both are used to form the metallic coating.

The metallic coating is generally thin in comparison to the scale of the drug particle. For instance, the volume of the metallic coating is typically 4% or less of the total volume of the micro particle; i.e., the drug particle being at least 96% by volume. Usually the volume of the metallic coating is even less, such as 3% or less, or 2% or less, and is frequently in the range of 0.5 to 2.0% by volume of the micro particle. In terms of coating thickness, the average thickness of the metallic coating on a drug particle is typically in the range of 1-25 nm, more typically 1-10 nm, and in some embodiments about 4 to about 6 nm. The thickness is typically measured via scanning electron microscopy (SEM) but may be measured by transmission electron microscopy (TEM). The thickness need not be uniform. In fact, parts of the drug particle surface may have no metallic coating. The metallic coating is often comprised of grains of the metal. The grains can be large, meaning that the grain size is comparable to the coating thickness, or small as in less than the coating thickness in dimension. Generally a large grain size is 5 nm or greater. With large grains, the metallic coating may be essentially a single grain thick. In contrast, with small grains, several grains may be necessary to reach the same coating thickness. Small grains are typically 0.25-3 nm. The use of small grains generally makes the metallic coating denser, which may increase the delay in drug release.

The composition, thickness, grain size, etc. of the metallic coating can be adjusted to provide the desired release characteristics for a given drug from a drug particle. Metallic coatings of this scale are generally not water proof; e.g., a 5 nm gold foil is water permeable. By limiting the drug particle's access to water (body fluids), the dissolution and/or transport of the drug from the drug particle into cells of the body can be delayed, controlled, or prolonged. An effect will be present if the metallic coating is uneven. For instance, a non-uniform coating that leaves 30% of the drug particle surface uncovered, can still delay the dissolution of the drug as dissolution under the covered surface areas can be impeded. In addition to providing a protective and/or release modifying barrier around the drug particle, the composition of the metallic coating itself can provide helpful effects. For instance, a silver coating may provide antimicrobial activity.

A plurality of micro particles is typically employed to achieve the desired dose amount of the biologically effective agent. In the dry state, such a plurality forms a powder. A powder that contains a plurality of micro particles requires two or more micro particles as described above, but may also include particles that are outside of the above dimensions and compositions. For example, a powder comprising a plurality of micro particles may include a micro particle that has a drug particle of 12 microns or a drug particle that has no metallic coating. Typically a powder according to the present invention does not merely comprise a plurality of micro particles, but comprises (or consists only of) a population of micro particles. A “population of micro particles” according to the invention means that the average drug particle size is within the range of 1-10 microns, optionally 2-5 microns, and, on average, has a metallic coating on the drug particles, which coating contains at least one metal. The micro particle population of the invention preferably has an average compositional and dimensional value as described above for the single micro particle. For example, the population may have an average drug particle composition of 50% excipient and 50% API, even though individual species of the micro particle from the population could have 80% excipient and 20% API, or 10% excipient and 90% API, etc. Such variations are typically a result of the manufacturing process. Usually more uniform, monodisperse populations are desired having smaller standard deviations (e.g., d₉₀ of 1-10 microns, optionally d₉₀ of 3-8 microns, etc.), but are not necessarily required. Likewise, the metallic coating in the population usually comprises 4% or less, typically 3% or less, and often 2% or less, including 0.5 to 2%, by volume based on the total volume of the micro particle population. In terms of average thickness, the metallic coating is often within the range of 1-25 nm, and typically 1-10 nm. In some embodiments, the average thickness is 4-6 nm. The average composition and grain size of the metallic coating can be within the above compositions and sizes as described for the individual micro particles, however the average grain sizes are typically within the range of 0.25-3.0 nm, often within 0.5-2.5 nm, and preferably 0.5-1.5 nm.

The micro particles of the present invention can be formulated into a variety of dosage forms. Examples of formulations include parenteral such as subcutaneous, intramuscular, or intravenous injection formulations; oral dosage forms such as capsules or liquid suspensions; and inhaled dosage forms including formulations for nebulizers or inhalers. The dosage forms contain a plurality of the micro particles, preferably at least one population of micro particles, according to the present invention. In some embodiments, the dosage form may contain a population of micro particles according to the invention and additional uncoated drug particles of the same or different biologically active agent. In some embodiments two or more populations of micro particles according to the invention may be used. The populations may differ by composition, such as using two different API's or using two different metallic coatings; or may differ by average value, such as average metallic coating volumes.

The use of different particles is a convenient way to provide combination therapy formulations. As the effective dose may differ greatly between actives, limiting the micro particles or drug particles to a single active allows the dosing to be controlled by the ratio of different micro particles and/or drug particles. The combinations of active are not particularly limited and include two or more of any of the actives identified above, especially those kinds of actives previously described for treating respiratory and/or lung disorders. A specifically contemplated combination involves a short or long acting β adrenergic receptor agonist and a corticosteroid. One or both of the drug-containing particles contains the metallic coating and is a micro particle of the invention. The combination of micro particles containing a short acting β adrenergic receptor agonist, such as albuterol, with uncoated drug particles that contain a corticosteroid, such as budesonide, fluticasone, or prednisone, is a specifically contemplated combination. In this embodiment, the metallic coating is preferably designed to achieve sustained release of the short acting agonist while the uncoated corticosteroid drug particle does not need any extension of release due to its longer duration of activity. For clarity, the uncoated drug particles preferably have the same compositional and dimensional features and requirements as described above for the drug particles used to make the micro particles of the invention, but fail to have a metallic coating. In another embodiment, both the agonist and the corticosteroid are in the form of micro particles of the present invention, e.g., two populations of micro particles with one containing the agonist and the other the corticosteroid.

A powder comprising the micro particles of the invention is often useful as a dosage form itself, or as an intermediate for making a final dosage from. For example, the powder, which may contain additional excipients and optionally other or additional biologically effective agent(s), can be formed into granules suitable for filling into capsules for oral administration. Alternatively, such a powder can be combined with an aqueous carrier, such as a saline solution, to form a parenteral formulation. A preferred use, however, is providing the powder for inhalable administration. Conventionally inhalable administration is involves a propellant gas driving a suspension of particles. In a dry method, such as with a conventional inhaler, the propellant gas drives the solid medicine-containing particles. In the present invention the solids would include a powder comprising a plurality of the micro particles according to the invention. A wet method, such as with a conventional nebulizer, combines the dry powder with water and disperses the resulting fine water droplets in a gas stream, typically of air. In both cases the dispersion of solids and/or drops in a gas forms an aerosol. A preferred powder of the present invention is suitable for aerosol administration, either directly or with the addition of water.

A pharmaceutical formulation that contains a plurality of micro particles according to the invention and optionally pharmaceutically acceptable excipient(s) is formulated to provide an effective amount of the active(s). Typically the formulation is administered as a unit dose one or more times per day to provide the effective amount. A unit dose is thus the single administration dose, e.g., one or two capsules, one or two inhalations, etc., which may be given or more times per day depending on the active. The content and ratio of micro particles in a pharmaceutical formulation is typically selected so that a unit dose can be prepared or dispensed. For example, a pharmaceutical composition may be prepared that comprises (i) micro particles of a corticosteroid such as budesonide and/or (ii) micro particles of albuterol or other β adrenergic receptor agonist, wherein a unit dose of the composition provides 600 micrograms of the albuterol or other agonist and/or 400 micrograms of budesonide or the therapeutic dose of another corticosteroid. In one embodiment, the drug particles have an average diameter of 5 microns and a gold coating having an average thickness of 5 nm, whether as a single drug formulation or a combination product. In another embodiment, both a corticosteroid and an agonist are present but only the albuterol or other β adrenergic receptor agonist is a micro particle. The corticosteroid, though a drug particle, does not have a metallic coating. In this alternative embodiment, both drug particles have an average diameter of 5 microns and the albuterol (or other agonist) drug particle has a gold coating with an average thickness of 5 nm, for example. By controlling the ratio and amount of micro particles (and optionally drug particles) and optionally pharmaceutically acceptable excipients in the formulation, an effective amount of the active can be dispensed as a unit dose.

The micro particle of the invention can be made by a process that comprises physical vapor deposition (“PVD”) or chemical vapor deposition (“CVD”). The coating of fine particles, albeit not drug particles, by PVD has been suggested in U.S. Pat. Nos. 8,354,355; 8,372,416; 8,618,020; and 8,728,390, each of which incorporated herein by reference.

A preferred method of making the micro particles comprises forming a metallic coating on a drug particle by a physical vapor deposition process, wherein the drug particle has a particle size of 1-10 microns and comprises a biologically effective agent and wherein the metallic coating comprises at least one metal. The physical vapor deposition process can be ion beam sputtering, magnetron sputtering, or evaporative sputtering such as from an ion or electron beam. The metallic coating is typically applied to a volume or thickness as described above; i.e., 4 vol. % or less, or an average thickness of 1-25 nm; etc. The drug particles are typically formed by forming a solution or suspension of biologically effective agent(s) in a solvent followed by evaporating the solvent under conditions to form the drug particle having a particle size of 1 to 10 microns. The evaporation step is often performed by use of an atomizer.

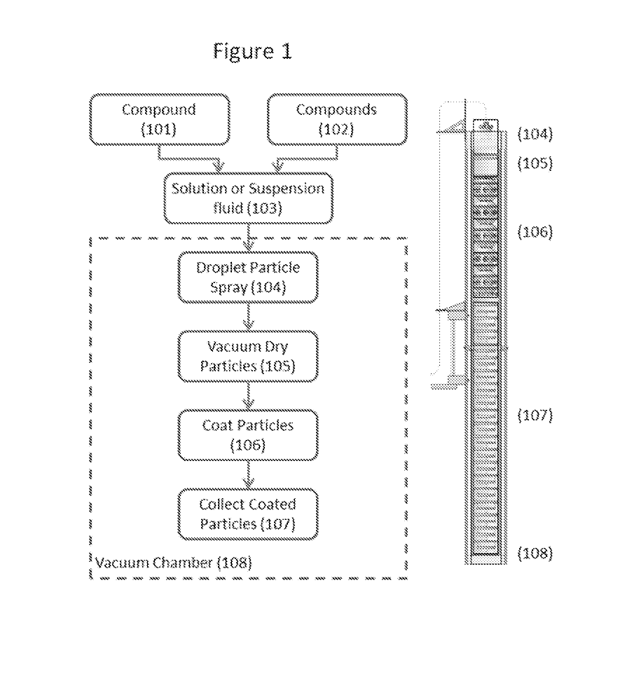

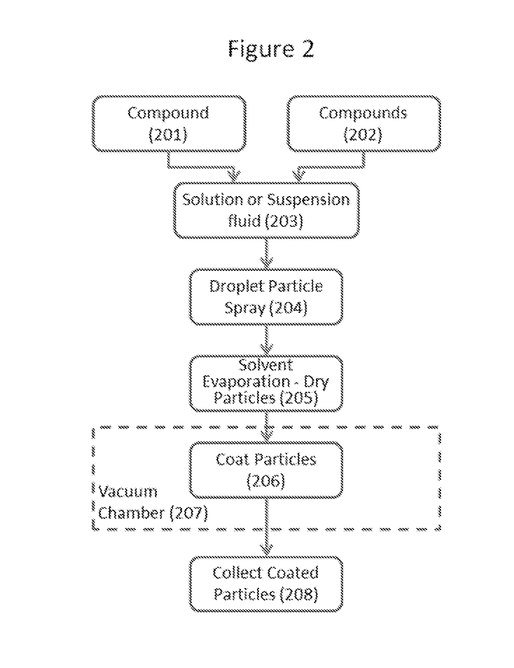

FIGS. 1-5 illustrate a preferred embodiment of the method of making the micro particles. FIG. 1 is a flow diagram of a first process for making the micro particles. A compound such as a biologically effective agent or excipient is provided by compound feeds 101 and/or 102 to a mixer 103. Though two feed sources are shown here, the process may have only one feed source or may have multiple feed sources, depending on the intended composition or use of the apparatus. The process as shown is suitable for making a micro particle having two biologically effective agents, either sequentially or simultaneously, i.e., two agents in a single drug particle. The compound(s) are mixed with a solvent, such as water, to form a solution or suspension fluid in mixer 103. The fluid is brought into a vacuum chamber 108 via the droplet particle sprayer (or atomizer) 104. The solvent is evaporated by vacuum to dry the droplets in step 105 to form particles from the compound(s). The particles from vacuum drying 105 are coated with a metal coating by physical vapor deposition in one or more PVD chambers 106 to form the micro particles of the invention. Once formed, the micro particles are collected at step 107 by passing through a circuitous route through varying filter sizes. Typically the route comprises vanes or plates having saw teeth to catch or trap the micro particles; though other filter designs can be used such as sequential meshes to trap or filter particles of a desired size range. The largest sized particles are generally trapped first and as the micro particles mover through the route, smaller sizes are caught/trapped. In this design, micro particles that are smaller than the desired size can be permitted to escape the collection zone and be carried away by the vacuum. Variations of the process shown in FIG. 1 are also contemplated. For example, the formation of the drug particles via an atomizer and drying can be conducted separately in a different vacuum and/or under non-vacuum conditions and then these pre-made drug particles can be brought into the coating step 106 within the vacuum 108 to form the micro particles. FIG. 2 illustrates a flow diagram of such a variation of FIG. 1, wherein particle formation is not conducted in a vacuum chamber. In this second flow diagram, again solute compound 201 and/or compounds 202 are mixed with a solvent, such as water, to form solution or suspension fluid 203. Though two feed sources are shown here, the process may have only one feed source or may have multiple feed sources, depending on the intended composition. The fluid is formed into a droplet spray by atomizer 204, but this occurs without using the vacuum chamber of the metallic coating step. The solvent evaporates to dry the droplets in step 205 to form particles from the compound 201 or compounds 202. The particles from step 205 are introduced into the vacuum chamber 207 and are coated by one or more physical vapor deposition treatments in step 206. The formed micro particles are collected in step 208, using any suitable means such as those described above for collection step 107. Other variations on the general theme of generating and coating micro particles can be envisioned.

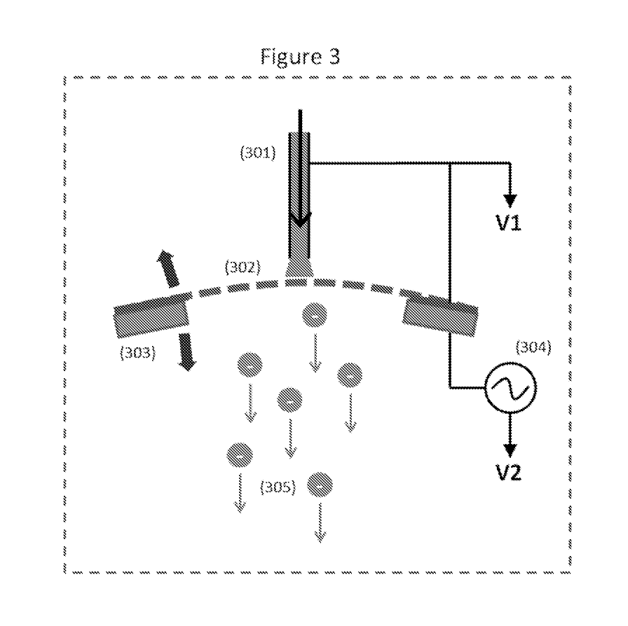

FIG. 3 shows a droplet particle sprayer 104. A capillary 301 is in proximity to a mesh 302 and both are subjected to a voltage V1. A piezoelectric crystal 303 is connected to a modulating voltage V2 from voltage source 304. The crystal 303 vibrates the mesh 302 at the frequency of the modulated voltage V2. The voltage V2, though variable, provides a net negative voltage in this example. Fluid from capillary 301 passing through mesh 302 is formed into a charged droplet spray 305. The capillary 301 provides a narrowed opening so as to reduce /eliminate over spray. While the jetting of the liquid into the vacuum would cause droplets to form, the use of the mesh helps to control the particle size. The holes in the mesh are typically 2-20 microns in size and more typically are in the range of 4-8 microns. The piezoelectric device shown is commercially available as a piezoelectric atomizer.

After forming the droplets, the liquid is evaporated by means of sufficient residence time in the vacuum chamber. This can be the same chamber as the atomizer or a separate/new chamber.

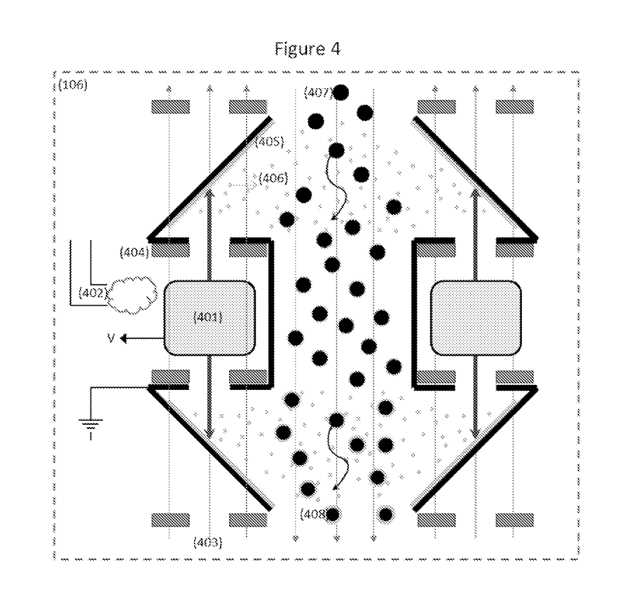

FIG. 4 shows the PVD coating of the dried drug particles. Ion generator 401 operates in a low pressure vacuum (e.g., 1-5 Torr) with ambient gas (such as Ar₂) from source 402 to generate energetic ions. The ions from the generator are accelerated by an electric potential V along magnetic field lines 403 from magnets 404 towards (metal) target 405 to generate the physical vapor material (such as Au) 406 from target 405. The ejected material 406 from target 405 deposits onto drug particles 407 to form coated particles 408. The material target 405 corresponds to the composition of the metallic coating. In practice, more than one PVD chamber as shown in FIG. 4 can be used in sequential fashion, such as using 2 chambers or as many as 10 chambers, though typically 3-6 chambers is likely to be sufficient, depending on the operating conditions and the physical size of the chamber. When sequential chambers are used, the input drug particle 407 becomes the partially coated drug particle produced from the previous chamber. In some embodiments, the target 405 may also be electrically biased so that charged ejected particles are not deposited but rather recaptured on the plate. This may increase the yield.

The ultimately coated particles are collected by any suitable means. Generally a circuitous route is used having many plates and diminishing sizes as mentioned above. The collected powder contains a plurality of micro particles of the invention and typically is a single population of micro particles meeting the above described composition and dimensional characteristics.

The entirety of the vacuum chamber operations from sprayer 104 through collection 107 can be performed in a vertical column or tower arrangement with the liquid being introduced at the top of the column and passing downward through the various stages. Though shown as a single vacuum chamber 108, the vacuum need not be constant throughout. For example, a different vacuum may be used in the vacuum drying section than in the PVD chamber. Collection can be in batch mode, wherein once the batch is complete, the collection filters are removed and tipped upside down to recover the micro particle powder.

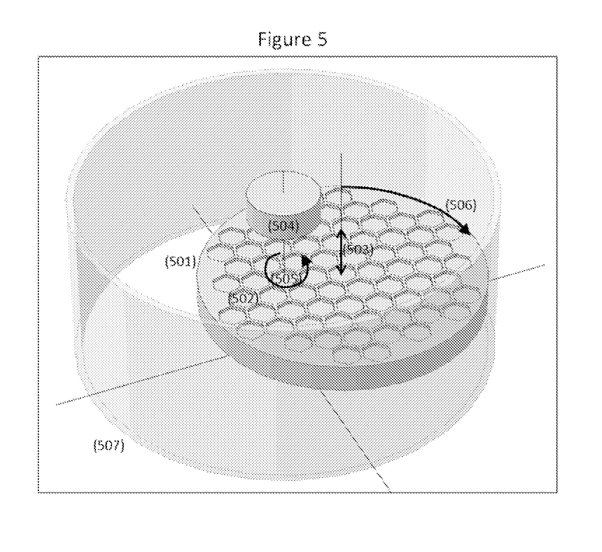

The PVD coating process of FIG. 4 is well suited for process step 106 of the diagram shown in FIG. 1. FIG. 5 shows a PVD coating method well suited for process step 206 shown in FIG. 2. Micro particles from step 205 are placed onto tray 501 which can be segmented into areas 502 to contain said micro particles. The tray 501 is vibrated as indicated by 503 to randomly orient and mix the particles. The particles are coated by physical vapor deposition (PVD) from target 504 which can be a magnetron sputtered target, radio frequency plasma sputtered target, or ion or electron beam sputtered target. To improve coating uniformity tray 501 can be moved in a planetary orbit 505 about 504 while rotating about as shown by 506. Said process can operate in a low pressure vacuum with ambient gas (such as Ar₂) or high vacuum in chamber 507.

The micro particles of the invention can be used to treat a variety of diseases or conditions. In particular treating respiratory ailments is contemplated. These include asthma, COPD, cystic fibrosis, emphysema, and lung cancers.

Each of the patents and articles mentioned above are incorporated herein by reference in their entirety. In view of the description of the invention, it will be readily apparent to the worker skilled in the art that the same may be varied in many ways without departing from the spirit of the invention and all such modifications are included within the scope of the present invention as set forth in the following claims.

1. A micro particle, comprising a drug particle having a size within the range of 1-10 microns and a metallic coating overlying said drug particle;
wherein said drug particle comprises a biologically effective agent; and
wherein said metallic coating comprises at least one metal selected from the transition metals, alkaline earth metals, and alkali metals.

2. The micro particle according to claim 1, wherein said metallic coating is 1-25 nanometers thick, such as 1-10 nanometers.

3. The micro particle according to claim 1, wherein said metallic coating comprises a metal selected from the group consisting of gold, silver, platinum, palladium, copper, nickel, and combinations thereof.

4. The micro particle according to claim 3, wherein said metallic coating is gold or silver.

5. The micro particle according to claim 4, wherein said metallic coating has a thickness of about 4 to about 6 nanometers.

6. The micro particle according to claim 1, wherein said biologically effective agent is an active pharmaceutical agent.

7. The micro particle according to claim 6, wherein said active pharmaceutical agent is a bronchodilator such as albuterol, formoterol or salmetrol; or anti-inflammatory agent such as fluticasone, budesonide, beclomethasone, mometasone, or ciclesonide.

8. The micro particle according to claim 1, wherein said biologically effective agent comprises at least 85% by weight of said drug particle.

9. A powder, which comprises a plurality of micro particles according to claim 1.

10. A method, which comprises forming a metallic coating on a drug particle by a physical deposition process, wherein said drug particle has a particle size of 1-10 microns and comprises a biologically effective agent and wherein said the metallic coating comprises at least one metal selected from the transition metals, alkaline earth metals, and alkali metals.

11. The method according to claim 10, wherein said physical vapor deposition process is ion beam sputtering, magnetron sputtering, or evaporative sputtering.

12. The method according to claim 10, wherein said metallic coating is applied to a thickness of 1 to 10 nanometers.

13. The method according to claim 10, wherein said metallic coating is gold, silver, platinum, palladium, copper, nickel, or a combination of two or more thereof.

14. The method according to claim 10, wherein said biologically effective agent comprises at least 85% of said drug particle.

15. The method according to claim 10, which further comprises forming a solution or suspension of said biologically effective agent in a solvent; and evaporating the solvent under conditions to form said drug particle having a particle size of 1 to 10 microns.
